(19) United States
(12) Patent Application Publication
(10) Pub. No.: US 2019/0343987 A1
(43) Pub. Date: Nov. 14, 2019

(54) LUBRICIOUS COATING COMPOSITIONS

(71) Applicant: BOSTON SCIENTIFIC SCIMED, INC., MAPLE GROVE, MN (US)

(72) Inventors: KENDAHL BENNIS, MINNETONKA, MN (US); JAN SEPPALA, LORETTO, MN (US); JEFFREY SCHNEIDER, CHAMPLIN, MN (US)

(73) Assignee: BOSTON SCIENTIFIC SCIMED, INC., MAPLE GROVE, MN (US)

(21) Appl. No.: 16/408,434

(22) Filed: May 9, 2019

Related U.S. Application Data
(60) Provisional application No. 62/670,679, filed on May 11, 2018.

(51) Int. Cl. A61L 27/34; A61L 27/54; A61F 2/02

(52) U.S. Cl. CPC A61L 27/34; A61L 27/54; C08L 39/06; A61L 2400/10; A61F 2/02

(57) ABSTRACT

In one aspect, the present disclosure pertains to lubricous coating compositions that comprise (a) a higher molecular weight polyvinylpyrrolidone, (b) a lower molecular weight polyvinylpyrrolidone, (c) a copolymer of sodium acrylate and acrylamide or a copolymer of acrylic acid and acrylamide, and (d) a polyfunctional unsaturated crosslinking agent. In some embodiments, such lubricous coating compositions are present in crosslinked form on the surface of a medical article. In some embodiments, the lubricous coating compositions further comprise a solvent, in which case such compositions may be, for example, applied to a substrate in the form of a layer and subsequently crosslinked, thereby forming a lubricious coating on the substrate.

### CROSS-REFERENCE TO RELATED APPLICATIONS

This application claims the benefit of priority under 35 U.S.C. § 119 of U.S. Provisional Application No. 62/670,679, filed May 11, 2018, the entire disclosure of which is hereby incorporated by reference.

### TECHNICAL FIELD

The disclosure is directed to lubricious coatings. More particularly, the disclosure is directed to lubricious coatings for medical devices.

### BACKGROUND

A wide variety of intracorporeal medical devices have been developed for medical use, for example, surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. There is an ongoing need to provide alternative medical devices as well as alternative methods for manufacturing and/or using medical devices. For example, as minimally invasive surgical techniques have improved, it has become increasingly common to insert and retrieve medical devices through catheters and the like having considerable length. Accordingly, it is desirable to minimize friction between the catheters that carry such devices and the devices themselves as well as with tissue with which they may come in contact. In the past, the industry has employed various hydrophobic oils and coatings such as olive oil, silicone, and the like as lubricants. Hydrophilic coatings, particularly hydrogels, also have been employed to impart lubricity to a variety of medical devices.

### SUMMARY

In a first example, a lubricous coating may comprise (a) a higher molecular weight polyvinylpyrrolidone (PVP), (b) a lower molecular weight polyvinylpyrrolidone (PVP), (c) a copolymer of sodium acrylate and acrylamide or a copolymer of acrylic acid and acrylamide, and (d) a cross-linked polymer forming a semi-interpenetrating network.

Alternatively or additionally to any of the examples above, in another example, the higher molecular weight PVP, the lower molecular weight PVP, and the copolymer of sodium acrylate and acrylamide or the copolymer of acrylic acid and acrylamide may be positioned within interstitial spaces of the semi-interpenetrating network of the cross-linked polymer.

Alternatively or additionally to any of the examples above, in another example, the higher molecular weight PVP may have a K-value ranging from 85 to 95 and the lower molecular weight PVP may have a K-value ranging from 15 to 32.

Alternatively or additionally to any of the examples above, in another example, the weight ratio of higher molecular weight polyvinylpyrrolidone to lower molecular weight polyvinylpyrrolidone may range from about 70:30 to about 90:10.

Alternatively or additionally to any of the examples above, in another example, the crosslinked polymer may be formed from a polyfunctional ethylenically unsaturated monomer selected from a polyfunctional acrylate compound, a polyfunctional methacrylate compound and a polyfunctional vinyl compound.

Alternatively or additionally to any of the examples above, in another example, the crosslinked polymer may be formed from neopentyl glycol diacrylate.

Alternatively or additionally to any of the examples above, in another example, the weight ratio of total polyvinylpyrrolidone to neopentyl glycol diacrylate may range from about 0.5:1 to about 4:1.

In another example, a medical article may comprise a medical article substrate that is at least partially covered by a layer of the lubricous coating composition in accordance any of the examples described herein.

Alternatively or additionally to any of the examples above, in another example, the layer may range from 0.1 to 20 micrometers in thickness.

In another example a lubricous coating composition may comprise (a) a higher molecular weight polyvinylpyrrolidone (PVP), (b) a lower molecular weight polyvinylpyrrolidone (PVP), (c) a copolymer of sodium acrylate and acrylamide or a copolymer of acrylic acid and acrylamide, (d) a cross-linking agent, and (e) a solvent.

Alternatively or additionally to any of the examples above, in another example, the solvent may comprise isopropanol and water.

Alternatively or additionally to any of the examples above, in another example, the weight ratio of water to isopropanol may range from about 50:50 to 10:90.

Alternatively or additionally to any of the examples above, in another example, the copolymer of sodium acrylate and acrylamide or the copolymer of acrylic acid and acrylamide may comprise about 0.05 to about 10% of the total solids.

Alternatively or additionally to any of the examples above, in another example, the composition may further comprise a therapeutic agent.

Alternatively or additionally to any of the examples above, in another example, the cross-linking agent may be neopentyl glycol diacrylate.

In another example, a lubricous coating may comprise (a) a higher molecular weight polyvinylpyrrolidone (PVP), (b) a lower molecular weight polyvinylpyrrolidone (PVP), (c) a copolymer of sodium acrylate and acrylamide or a copolymer of acrylic acid and acrylamide, and (d) a cross-linked polymer forming a semi-interpenetrating network

Alternatively or additionally to any of the examples above, in another example, the higher molecular weight PVP, the lower molecular weight PVP, and the copolymer of sodium acrylate and acrylamide or the copolymer of acrylic acid and acrylamide may be positioned within interstitial spaces of the semi-interpenetrating network of the cross-linked polymer.

Alternatively or additionally to any of the examples above, in another example, the higher molecular weight PVP may have a K-value ranging from about 85 to about 95 and wherein the lower molecular weight PVP may have a K-value ranging from about 15 to about 32.

Alternatively or additionally to any of the examples above, in another example, the weight ratio of higher molecular weight polyvinylpyrrolidone to lower molecular weight polyvinylpyrrolidone may range from about 70:30 to about 90:10.

Alternatively or additionally to any of the examples above, in another example, the crosslinked polymer may be formed from a polyfunctional ethylenically unsaturated monomer selected from a polyfunctional acrylate compound, a polyfunctional methacrylate compound and a polyfunctional vinyl compound.

Alternatively or additionally to any of the examples above, in another example, the crosslinked polymer may be formed from neopentyl glycol diacrylate.

Alternatively or additionally to any of the examples above, in another example, the weight ratio of total polyvinylpyrrolidone to neopentyl glycol diacrylate may range from about 0.5:1 to about 4:1.

In another example, a medical article may comprise a medical article substrate that is at least partially covered by a layer of the lubricous coating in accordance with any of the examples described herein.

Alternatively or additionally to any of the examples above, in another example, the layer may range from about 0.1 to about 20 micrometers in thickness.

Alternatively or additionally to any of the examples above, in another example, the medical article may be an implantable or insertable medical device.

In another example a lubricous coating composition may comprise (a) a higher molecular weight polyvinylpyrrolidone (PVP), (b) a lower molecular weight polyvinylpyrrolidone (PVP), (c) a copolymer of sodium acrylate and acrylamide or a copolymer of acrylic acid and acrylamide, (d) a cross-linking agent, and (e) a solvent.

Alternatively or additionally to any of the examples above, in another example, the solvent may comprise isopropanol and water.

Alternatively or additionally to any of the examples above, in another example, the weight ratio of water to isopropanol may range from about 50:50 to about 10:90.

Alternatively or additionally to any of the examples above, in another example, the total solids may be adjustable to control a thickness of the coating and particulate shedding

Alternatively or additionally to any of the examples above, in another example, the copolymer of sodium acrylate and acrylamide or the copolymer of acrylic acid and acrylamide may comprise about 0.05 to about 10% of the total solids.

Alternatively or additionally to any of the examples above, in another example, the composition may further comprise a therapeutic agent.

In another example, a method may comprise applying the lubricous coating composition of any of the above examples to a substrate in the form of a layer, removing at least a portion of the solvent, and crosslinking the composition by applying of UV light.

Alternatively or additionally to any of the examples above, in another example, the method may further comprise sterilizing the substrate.

Alternatively or additionally to any of the examples above, in another example, the article is a medical article comprising a lubricious coating.

Alternatively or additionally to any of the examples above, in another example, the medical article may be an implantable or insertable medical device.

The above summary of some example embodiments is not intended to describe each disclosed embodiment or every implementation of the invention.

### BRIEF DESCRIPTION OF THE DRAWINGS

The disclosure may be more completely understood in consideration of the following detailed description of various embodiments in conjunction with the accompanying drawings, in which:

FIG. 1 illustrates the viscosity of various coating solutions at varying revolutions per minute (RPM);

FIG. 2 illustrates the lubricity and durability of various sterilized and unsterilized coating compositions;

FIG. 3 illustrates the viscosity of various coating solutions at varying revolutions per minute (RPM); and

FIG. 4 illustrates the lubricity and durability of various coating compositions. While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

It is generally known to provide substrates, for instance medical devices or parts of such devices, with a hydrophilic coating for the purpose of reducing the friction between medical devices themselves or between a medical device and a tissue when the device is introduced in an aqueous environment, such as the human body, or within another medical device. Such hydrophilic coatings have also been referred to as lubricous or “slippery” coatings. Catheters and other medical devices used for introduction in blood vessels, urethra, body conduits and the like and guide wires used with such devices are examples of articles which may be provided with hydrophilic coatings. Catheters for balloon angioplasty and biopsy are specific examples of such catheters. Other illustrative medical devices may include, but are not limited to, stents, embolic filters, implantable devices, treatment devices, diagnostic devices, guide catheters, sheaths, etc. In some cases, the hydrophilic coating may shed particulates as the device is advanced or positioned within the body. What may be desirable is a lubricious coating that decreases particulate burden (e.g., reduces particulates within the body attributable to the lubricious coating) for improved patient safety.

In one example, the present disclosure pertains to lubricous coating compositions for various articles. The coating may comprise a mixture of a higher molecular weight polyvinylpyrrolidone (higher MW PVP), a lower molecular weight polyvinylpyrrolidone (lower MW PVP), an acrylate copolymer or polyacrylic acid as well as an additional cross-linked polymer. Without being bound by theory, in some cases, during the curing process, the cross-linked polymer may form a semi-interpenetrating polymer network and the higher MW PVP, lower MW PVP, and the acrylate copolymer may be situated within the interstitial spaces of the semi-interpenetrating network. However, some (e.g., less than all of the higher MW PVP, lower MW PVP, and/or the acrylate copolymer or polyacrylic acid) may be cross-linked or otherwise bonded to the cross-linked polymer.

Because it is difficult to measure molecular weight of a sample of PVP directly, the K-value has been adopted to classify the molecular weight of PVP. The K-value is a function of the average degree of polymerization and intrinsic viscosity of a given polymer and is calculated from the kinematic viscosity of a 1% w/v aqueous solution of the polymer.

As used herein, a higher MW PVP is defined as one having a K-value between about 60 and about 95 (e.g., ranging from about 60 to about 95). This corresponds to a weight average molecular weight between approximately 100,000 and 1,300,000.

As used herein, a lower MW PVP is defined as one having a K-value between about 5 and about 35 (e.g., ranging from about 5 to about 35). This corresponds to a weight average molecular weight between approximately 2,000 and 95,000.

As used herein, the term “organic acid group” is meant to include any groupings which contain an organic acidic ionizable hydrogen. Examples of functional groupings which contain organic acidic ionizable hydrogen are the carboxylic and sulfonic acid groups. The expression “organic acid functional groups” is meant to include any groups which function in a similar manner to organic acid groups under the reaction conditions, for instance metal salts of such acid groups, particularly alkali metal salts like lithium, sodium and potassium salts, and alkaline earth metal salts like calcium or magnesium salts, and quaternary amine salts of such acid groups, particularly quaternary ammonium salts.

Examples of polyvinylpyrrolidone materials useful in the present disclosure include Povidone K12, Povidone K15, Povidone K17, Povidone K25, Povidone K30, Povidone K60, Povidone K90 and Povidone K120, among others. Polyvinylpyrrolidone is available from BASF Corp, Parsippany, N.J., USA under the tradenames Kollidon® and Luvitec® and from Ashland Inc., Halethorpe, Md., USA as Plasdone®.

In some embodiments, a weight ratio of higher MW PVP to lower MW PVP may be in the range of about 90:10 to about 50:50. For example, high MW PVP may account for anywhere in the range of about 50% to about 90% of the total weight of PVP while low MW PVP may account for anywhere in the range of about 10% to about 50% of the total weight of PVP in the lubricious coating.

Examples of an acrylate copolymer may include, but are not limited to sodium acrylate-acrylamide copolymer, other salt neutralized (e.g., potassium, lithium and ammonium) polyacrylic acid-acrylamide copolymers, or acrylic acid-acrylamide copolymers which are available from many different suppliers including, but not limited to Sigma-Aldrich®, St. Louis, Mo., USA, The Dow Chemical Company, Midland, Mich., USA, or BASF Corp, Parsippany, N.J., USA. In one example, Magnafloc® which is available from BASF Corp, Parsippany, N.J., USA, may be used. In some instances, Magnafloc® 525 may be one example of an illustrative acrylate copolymer.

Without wishing to be bound by theory, the acrylate copolymer or polyacrylic acid may improve processability (e.g., polymer solution rheology) and/or deliverability of the coated device. For example, the raw materials may be tailored to provide a balance of processability and/or performance. In one example, molecular weight and/or copolymer ratio of a polyacrylic acid-co-acrylamide may change moisture absorption and/or viscosity. Viscosity may be changed by polymer selection or formulation. Polyacrylic acid may be modified with a base to change solubility in solution. The base may be volatile to delay acid functionality in solution yet obtain acid functionality in the coating. Salts may be added to block or delay cross-linking reactions. Lubricity may be tailored by adjusting moisture absorption with block length, degree of ionization in solution, or post treatment of the coating with a base. It is further completed that when dried these hydrophilic polymers become lubricious upon contact with an aqueous solution.

In some embodiments, the polyacrylic acid polymers, sodium acrylate-acrylamide copolymer, or acrylic acid-acrylamide copolymers may be about 0.05 to about 50%, about 0.1 to about 5%, or about 0.5% to about 1.5% of the total solids of the composition.

Depending on the composition, the crosslinking polymer may have a degree of crosslinking less than 5%, a degree of crosslinking between 5% and 95%, or a degree of crosslinking greater than 99%. The degree of crosslinking may range from 1% or less to 2% to 5% to 10% to 25% to 50% to 75% to 90% to 95% to 98% to 99% or more. As will be described in more detail herein, in some cases, the crosslinking polymer may be at least partially crosslinked using an ultraviolet (UV) light source.

In some embodiments, the lubricous coating compositions are applied to the surface of a medical article in solid form, with the crosslinked polymer being at least partially crosslinked. In other embodiments, the lubricous coating compositions are applied in a liquid form, with the crosslinking polymer being substantially uncrosslinked. Such compositions may be, for example, applied to a substrate in the form of a layer and then at least partially crosslinked, thereby forming a lubricious coating on the substrate. In some embodiments, such compositions may comprise a solvent, which may be removed before, during and/or after a crosslinking step.

Polyfunctional ethylenically unsaturated monomers may be used to form the crosslinked polymer having a semi-interpenetrating network. The polyfunctional ethylenically unsaturated monomers are compounds, including monomeric and oligomeric compounds, that have two or more ethylenically unsaturated groups thereon that may be readily polymerized by a radical mechanism to form a polymer. Typically, such compounds have a molecular weight number average of about 5000 or less, more typically about 1000 or less. Suitable polyfunctional ethylenically unsaturated monomers include di- and tri-functional acrylate and methacrylate compounds, collectively referred to as (meth)acrylate compounds, including (meth)acrylate esters, as well as divinyl and trivinyl compounds. Specific examples of polyfunctional ethylenically unsaturated monomers that may be used in the compositions of the present disclosure include neopentyl glycol di(meth)acrylates, including neopentyl glycol diacrylate (NPGDA), ethylene glycol di(meth)acrylates, 1,3-propylene glycol di(meth)acrylates, 1,4-butanediol di(meth)acrylates, 1,6-hexanediol di(meth)acrylates, diethylene glycol di(meth)acrylates, triethylene glycol di(meth)acrylates, tetra ethylene glycol di(meth)acrylates, and polyethylene glycol di(meth)acrylates. In some embodiments the ethylenically unsaturated monomers that may be employed are alkoxylated and include ethoxylated and propoxylated (meth)acrylates.

Without being bound by theory, it is believed that the polyfunctional ethylenically unsaturated monomer(s) are crosslinked upon exposure to heat or actinic radiation such as UV irradiation, whereupon the crosslinked polyfunctional ethylenically unsaturated monomer(s) acts like a mesh that holds at least a portion of the PVP and/or acrylate copolymer and/or polyacrylic acid in place (such a system is known as a semi-interpenetrating polymer network). For example, it is contemplated that the PVP and/or acrylate copolymer and/or polyacrylic acid may be positioned within the interstitial spaces of the semi-interpenetrating polymer network. In some instances, some of the PVP and/or acrylate copolymer and/or polyacrylic acid may chemically bond with one or more of the polyfunctional ethylenically unsaturated monomer(s), the PVP, and/or acrylate copolymer and/or polyacrylic acid. Such a cross-linking polymer may allow the lubricious coating to be coated onto medical devices and cured with ultraviolet (UV) light.

In some embodiments, a weight ratio of total polyvinylpyrrolidone (PVP) to polyfunctional ethylenically unsaturated monomer(s) may be in the range of about 0.5:1 to about 4:1.

Monofunctional ethylenically unsaturated monomers may also be optionally included in some compositions (and thus excluded in other compositions). Examples include mono(meth)acrylate esters, mono-vinyl compounds, and so forth.

An additional hydrophilic polymer may also be optionally included in some compositions (and thus excluded in other compositions). Examples of such polymers, which may be included (or excluded), include polyethylene glycol, polypropylene glycol, polyvinylpryrrolidone, hydrophilic urethane polymers, including acrylated urethanes, and so forth. The polymer may comprise monomer units from one or more monomers having organic acid functional groups. Examples of such monomers include acrylic acid, methacrylic acid, and isocrotonic acid.

A free radical initiator may also be optionally included in some compositions of the present disclosure (and thus excluded in other compositions). The free radical initiator may be, for example, a photoinitiator. Non-limiting examples of free radical photoinitiators that may be employed include benzophenones, ketones, acrylated amine synergists, alpha-amino ketones, acyl phosphine oxides including bis-acyl phosphine oxides, and benzil ketals. More specific examples of photoinitiators suitable for use herein include, but are not limited to, 2-phenyl-1-indanone; IRGACURE 184 from Ciba Specialty Chemicals, BENACURE 184 from Mayzo and SARCURE SR1122 from Sartomer, all of which are 1-hydroxylcyclohexylphenyl ketone (HCPK) initiators; BENACURE BP benzophenone; BENACURE 651 and IRGACURE 651, both of which are benzil dimethyl ketal or 2,2′dimethoxy-2-phenylacetophenone; BENACURE 1732 hydroxy-2-methyl-1-phenyl-1-propanone; IRGACURE 819 bis(2,4,6-trimethylbenzoyl)-phenylphosphineoxide, IRGACURE 907 2-Methyl-1-[4-(methylthio)phenyl]-2-(4-morpholinyl)-1-propanone; IRGACURE 369 morpholinoketone; and so forth and blends thereof. Photoinitiators are also available commercially in a variety of blends. Examples of commercially available blends include, but are not limited to, SARCURE SR1136 is a blend of 4-methylbenzophenone and benzophenone; SARCURE SR1137 is a blend of trimethylbenzophenone and methylbenzophenone; and BENACURE 500, a blend of 1-hydroxylcyclohexylphenyl ketone and benzophenone.

Other optional additives may be employed in the coating compositions of the present disclosure including, but not limited to, flow or viscosity modifiers, antioxidants, coupling agents, surfactants, therapeutic agents, and so forth. Any such additives may be typically incorporated into the composition at levels of 10% or less (e.g., ranging from 10% to 5% to 2% to 1% to 0.5% or less), based on the dry weight (e.g., excluding solvent) of the composition.

In preparing liquid coating compositions for application to a substrate, the higher and lower molecular weight polyvinylpyrrolidones, and sodium acrylate-acrylamide copolymer (or polyacrylic acid polymers) are suitably mixed with polyfunctional unsaturated crosslinking agent in a solvent that contains one or more solvent species. Examples of suitable solvents species include, but are not limited to, water and organic solvents including lower alcohols such as methanol, ethanol, and isopropyl alcohol (IPA), linear or cyclic carboxamides such as N,N-dimethylacetamide (DMAC), N,N-diethylacetamide, dimethylformamide (DMF), ethyl formamide, diethylformamide, N-methyl-2-pyrrolidone (NMP); dimethyl sulfoxide (DMSO), acetonitrile, acetone and acetyl acetone, acrylonitrile, benzonitriledimethyl acetamide, 1,4-dioxane, dipropyl sulfone, aromatic solvents such as toluene and xylene, nitrobenzene, phenylacetate, propionitrile, and so forth. In some cases, solvent species may be water soluble. Blends of solvent species such as those set forth above may be used. In one embodiment, isopropyl alcohol in combination with water acts as a suitable solvent. In many embodiments, the weight ratio of water to alcohol ranges from about 0:100 to about 70:30.

Typically, the liquid coating compositions for use in the present disclosure may contain from about 1% to about 10% solids, or about 2% to about 7% solids, or about 3% to about 6% solids. The solids of the liquid coating composition may include the higher MW PVP, the lower MW PVP, an acrylate copolymer or polyacrylic acid, and the cross-linking polymer.

The mixture of solvent and coating composition may be applied to the medical device by any method known in the art including, but not limited to, spraying, dipping, rolling, painting (e.g., brush painting, sponge painting, etc.), and so forth. The coating may then be allowed to dry, by evaporation of the solvent. The solvent may be more readily evaporated at an elevated temperature, although room temperature drying is typically acceptable.

A variety of substrate materials may be used in conjunction with the present disclosure including organic and inorganic substrates, typically polymer substrates, metal substrates and glass substrates, among others. Examples of metal substrates include pure metals such as platinum, gold, iridium and titanium, or metal alloys such as stainless steel including platinum enriched stainless steel (PERSS), Nitinol alloys, and cobalt chromium alloys.

Examples of polymer substrates include the following, among many others: (a) olefin homopolymers and copolymers, including homopolymers and copolymers of C2-C8 alkenes, for example, polyethylene and polypropylene, ethylene-vinyl acetate copolymers (EVA), and isobutylene-styrene copolymers, including block copolymers comprising one or more polystyrene blocks and one or more polyisobutylene blocks, for instance, poly(styrene-b-isobutylene-b-styrene) (SIBS), among others, (b) polyamides such as nylons, polyether-polyamide block copolymers such as poly(tetramethylene oxide-b-polyamide-12) block copolymer, available from Elf Atochem as PEBAX, among others, (c) fluoropolymers, including homopolymers and copolymers of C2-C8 alkenes in which one or more hydrogen atoms are substituted with fluorine, for example, polytetrafluoroethylene (PTFE), polyhexafluoropropene (PVDF), polyvinylidene fluoride (PVDF), and poly(vinylidene fluoride-co-hexafluoropropene) (PVDF-HFP), among others, (d) polyurethane copolymers, including copolymers that are polyether based, polyester based, polycarbonate based, aromatic based and aliphatic based, including polyisobutylene based polyurethanes (PIB-PU), among others, and (e) silicone homopolymers and copolymers (also referred to as polysiloxanes) such as polydimethylsiloxane.

Examples of substrates include medical article substrates, specific example of which include medical device substrates, for instance, implantable or insertable medical device substrates. A variety of devices may thus be partially or completely coated with compositions in accordance with the present disclosure, including, for example, catheters (e.g., renal or vascular catheters), balloons, catheter shafts, guide wires, filters (e.g., vena cava filters), stents (including coronary vascular stents, cerebral stents, urethral stents, ureteral stents, biliary stents, tracheal stents, gastrointestinal stents and esophageal stents), stent grafts, cerebral aneurysm filler coils (including Guglilmi detachable coils and metal coils), vascular grafts, myocardial plugs, patches, pacemakers and pacemaker leads, heart valves, vascular valves, tissue engineering scaffolds for cartilage, bone, skin and other in vivo tissue regeneration, and so forth.

In some embodiments, no primer layer or coupling agent may be applied to the substrate before the coating is applied. However, in some embodiments, the substrate may be treated with plasma or corona discharge before application of the coating composition. In other embodiments, a primer layer or coupling agent may be applied to the substrate before the coating is applied. Some illustrative primers or coupling agents may include, but are not limited to, polyurethanes, silanes, polyacrylic acid-polyethylene copolymers, etc.

Coating compositions in accordance with the present disclosure may be cured, for example, by exposing the coating composition to heat or actinic radiation such as UV light for a short period of time. This initiates polymerization/crosslinking of the ethylenically unsaturated monomer(s). The polyfunctionality of at least some of the ethylenically unsaturated monomer(s) produces a high degree of crosslinking upon polymerization. At least for compositions based on acrylate esters it is generally desirable to cure in a low oxygen atmosphere, such as under a blanket of nitrogen, helium or argon gas. The amount of time needed to cure the surface is dependent on the source of energy, the relative amounts of constituents in the composition, the thickness of the coating desired, and other factors. Generally, the amount of time required for thermal cure is from about 1 to 30 minutes. UV curing requires less time and can generally be in the range of about two minutes or less. Curing around and along the substrate can be accomplished by incrementally or continuously using irradiation from multiple angles using spaced lamps and/or reflectors; rotation of the substrate, light source or light beam; longitudinal movement of the substrate, light source or light beam; or a combination of such techniques. The polymerizable composition is typically cured by irradiation with a suitable source of activating radiation such as ultraviolet (UV) radiation. Light sources may be narrow or broad spectrum or laser beam sources. Suitably the composition is cured using a high intensity broad spectrum ultraviolet lamp such as mercury arc capillary lamps which have some output in the UVC region (280 nm-100 nm). In some embodiments the composition is photocured with UV lamps that are sequenced or pulsed in a way that allows for some heat dissipation during the curing cycle.

In some embodiments the crosslinked coating thickness on the substrate may be in the range of from 0.1 micrometers or less to 20 micrometers or more (e.g., from 0.1 to 0.2 to 0.5 to 1 to 2 to 5 to 10 to 20 micrometers), or about 0.1 to about 5 micrometers. The coating thickness will be affected by the percent solids in the coating and the technique of application, among other factors. Multiple coatings may be applied to achieve a desired coating thickness.

In some embodiments the coating compositions described herein may comprise a therapeutic agent, for example, selected from antimicrobial agents, antibiotic agents, anti-cancer agents, agents for treating calcifications, antirestenotic agents and antithrombotic agents, and combinations thereof, among others. The therapeutic agent(s) may be added to the coating composition prior to curing or applied onto the coating after it has been cured. Therapeutic agent(s) carried in the polymer coating may remain in the coating or elute out of the coating when the coating is wet, thereby delivering the therapeutic agent(s) to immediately adjacent areas of the body.

The present composition may reduce the particulate burden relative to previously known high-throughput UV-cured hydrophilic coatings. Shedding of hydrophilic coatings from a medical device may be measured using a bench model that simulates the vasculature to be navigated and typical procedural use. After the simulated vascular navigation, particles shed from the device may be measured in their hydrated state using light obscuration. Coating surface area is dependent on the device needs and varies per medical application. For example, device coating surface areas can range from 1.06 cm² to 38.52 cm² with potential to be larger or smaller based on medical application. It is contemplated for this present composition that a standard industry representation of a balloon catheter yields approximately a coating area of 9.5 cm². When the standard industry representation of a balloon catheter is coated with the present lubricious coating, advanced through the bench model, and particles shed analyzed, the present composition may have a total particulate burden for particulates having a size of 10 micrometers (μm) or greater of about 1,000 to about 10,000, a total particulate burden for particulates having a size of 25 micrometers (μall) or greater of about 0 to about 1,000, and a total particulate burden for particulates having a size of 50 micrometers (μm) or greater of about 0 to about 100. This may be a reduction in particulate burden in the range of about 30% to about 75% or more relative to previously known high-throughput UV-cured hydrophilic coatings.

It is further contemplated that the present composition may increase the lubricity of the coating relative to previously known high-throughput UV-cured hydrophilic coatings by about 50% to about 62%.

The invention is illustrated by the following non-limiting examples.

### Example 1

A first stock solution of lower MW PVP (having a K-value of 30), higher MW PVP (having a K-value of 90), and neopentyl glycol diacrylate (NPGDA) in isopropyl alcohol (IPA) was prepared using the following weight percentages:

IPA: 95.85%

NPGDA: 1.40%

K90 PVP: 1.91%

K30 PVP: 0.84%

The ingredients were mixed under proper agitation until thoroughly mixed.

A second stock solution of acrylic acid-acrylamide copolymer (e.g., Magnafloc® 525) and water was prepared using the following weight percentages:

Water: 98.50%

acrylic acid-acrylamide copolymer: 1.50%

The ingredients were mixed under proper agitation until thoroughly mixed.

The first stock solution and the second stock solution were then mixed to formulate a coating solution having varying percent solids of acrylic acid-acrylamide copolymer in the coating solution. Table 1 below illustrates the quantities of the first stock solution and the second stock solution utilized to form the coating solutions, as well as the percent of total solids of acrylic acid-acrylamide copolymer. The combined weight of the NPGDA, K90 PVP, K30 PVP, and acrylic acid-acrylamide copolymer represent the total solids.

TABLE 1
Coating Solutions with Varying Percent of Total
Solids of Acrylic Acid-Acrylamide Copolymer

| | Solution | Solution | Solution | Solution | Solution | Solution |
|---|---|---|---|---|---|---|
| | #1 | #2 | #3 | #4 | #5 | #6 |
| First Stock | 20.49 g | 20.49 g | 20.49 g | 20.49 g | 20.49 g | 20.49 g |
| Solution | | | | | | |
| Second Stock | 29.91 g | 29.82 g | 29.73 g | 29.64 g | 29.60 g | 29.55 g |
| Solution (acrylic | | | | | | |
| acid-acrylamide | | | | | | |
| copolymer ) | | | | | | |
| % solids of acrylic | 50% | 40% | 30% | 20% | 15% | 10% |
| acid-acrylamide | | | | | | |
| copolymer | | | | | | |

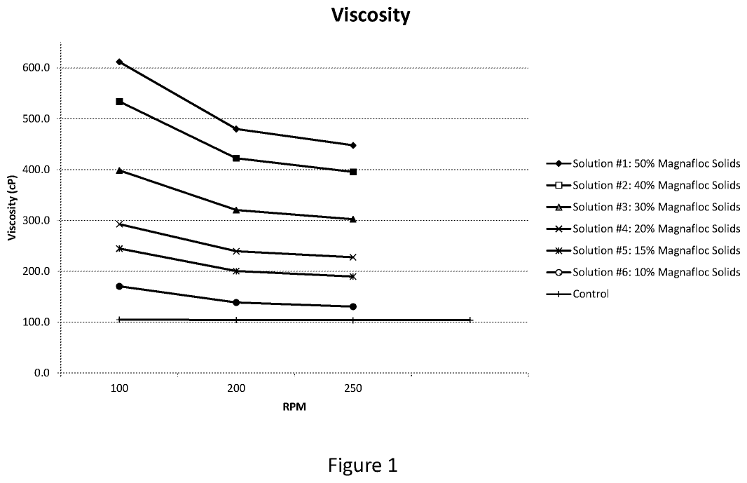

The viscosity of the solutions was then determined utilizing a viscometer. FIG. 1 illustrates the viscosity (in centipoise (cP)) of the above solutions at varying revolutions per minute (RPM) as well as a control solution to illustrate how the varying quantities of acrylic acid-acrylamide copolymer impacts the viscosity of the solution. The control solution is the first stock solution (e.g., having 0% acrylic acid-acrylamide copolymer). As can be seen, increasing the percentage of acrylic acid-acrylamide copolymer increases the viscosity of the coating solution.

### Example 2

Several coating compositions were prepared using the components and quantities listed in Table 2 below. The compositions were mixed such that the ratio of total PVP to acrylic acid-acrylamide copolymer remained constant for each formulation while varying the total percent solids of the compositions.

TABLE 2
Coating Solutions with Varying Percent of Total Solids

| Component | Form #1 | | Form #2 | | Form #3 | | Form #4 | |
|---|---|---|---|---|---|---|---|---|
| Isopropyl Alcohol | 39.3 | g | 58.40 | g | 57.8 | g | 58.9 | g |
| (IPA) | | | | | | | | |
| Water | 58.9 | g | 28.90 | g | 38.6 | g | 39.3 | g |
| neopentyl glycol | 0.57 | g | 0.85 | g | 1.14 | g | 0.57 | g |
| diacrylate | | | | | | | | |
| (NPGDA) | | | | | | | | |
| K90 PVP | 0.78 | g | 1.17 | g | 1.57 | g | 0.78 | g |
| K30 PVP | 0.35 | g | 0.52 | g | 0.69 | g | 0.35 | g |
| acrylic acid- | 0.10 | g | 0.15 | g | 0.20 | g | 0.30 | g |
| acrylamide | | | | | | | | |
| copolymer | | | | | | | | |

| | | | | |
|---|---|---|---|---|
| Percent Total | 1.8% | 2.7% | 3.6% | 2.0% |
| Solids | | | | |

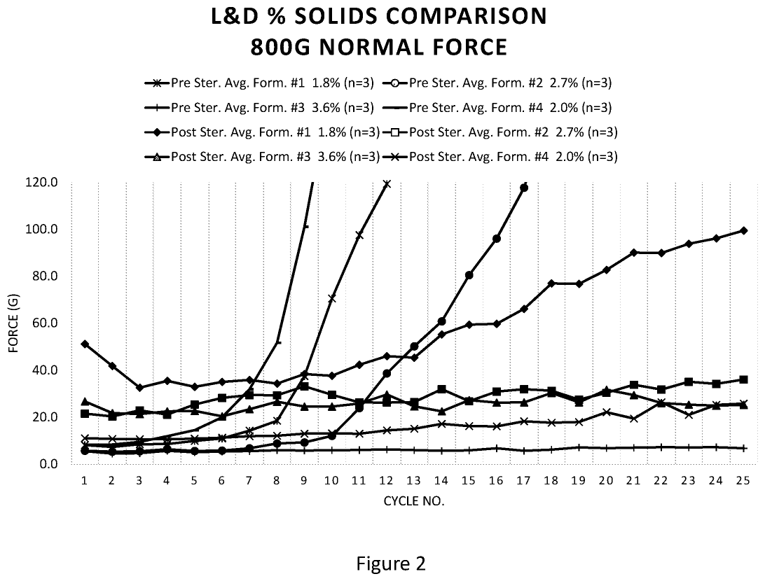

The compositions were then individually coated onto at least two catheter shafts and cured using UV irradiation. Half of the catheter shafts were tested for lubricity and durability following UV irradiation and half of the catheter shafts were sterilized after UV irradiation and then tested for lubricity and durability (L&D). To test for L&D, a mandrel inserted into the catheter shaft and the catheter shaft and mandrel secured within a clamping mechanism. A clamp was then closed about the catheter shaft at a normal force of about 800 grams (g). The force required to pull the catheter from the clamp is measured over a number of cycles. FIG. 2 illustrates a graph of the force required (in grams) to pull the catheter shaft from the clamp for each of the coating compositions. Each composition was tested without sterilization and with sterilization. As can be seen in FIG. 2, the samples that were sterilized exhibited a greater lubricity and durability than the unsterilized samples. Without wishing to be bound by theory, it is believed that the sterilization may facilitate reflow for a smoother surface as well as facilitating further cross-linking of the NPGDA.

### Example 3

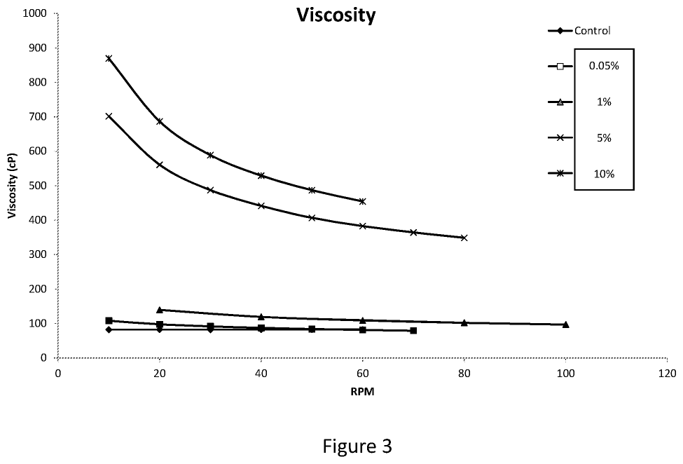

Several coating compositions were prepared using isopropyl alcohol (IPA), water, lower MW PVP (having a K-value of 30), higher MW PVP (having a K-value of 90), neopentyl glycol diacrylate (NPGDA), and an acrylic acid-acrylamide copolymer (e.g., Magnafloc® 525). The compositions were mixed such that the percent of total solids of the acrylic acid-acrylamide copolymer was 0% (e.g., the control solution), 0.05%, 1%, 5%, and 10%. The viscosity of the solutions was then determined utilizing a viscometer. FIG. 3 illustrates the viscosity (in centipoise (cP)) of the above solutions at varying revolutions per minute (RPM) as well as a control solution to illustrate how the varying the percent of total solids of an acrylic acid-acrylamide copolymer impacts the viscosity of the solution. As can be seen, increasing the percentage of an acrylic acid-acrylamide copolymer increases the viscosity of the coating solution.

### Example 4

Several coating compositions were prepared using isopropyl alcohol (IPA), water, lower MW PVP (having a K-value of 30), higher MW PVP (having a K-value of 90), neopentyl glycol diacrylate (NPGDA), and an acrylic acid-acrylamide copolymer (Magnafloc® 525). The compositions were mixed such that the percent of total solids of acrylic acid-acrylamide copolymer was 0.05%, 1%, and 5% with the lower MW PVP (having a K-value of 30), higher MW PVP (having a K-value of 90), neopentyl glycol diacrylate (NPGDA), and acrylic acid-acrylamide copolymer accounting for all of the solids.

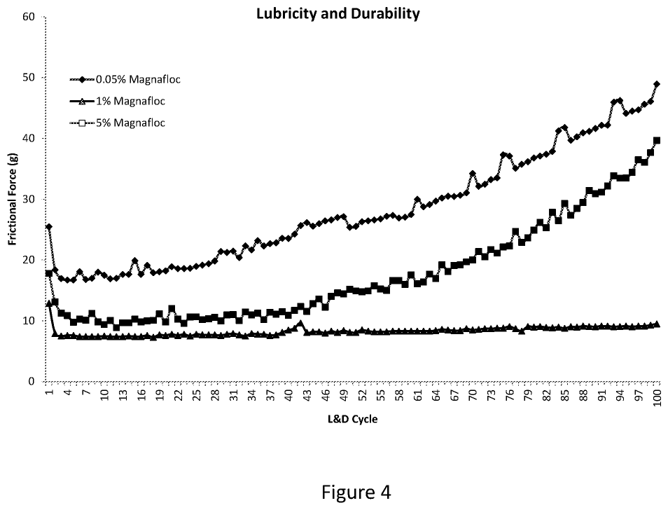

The compositions were then individually coated onto catheter shafts and cured using UV irradiation and sterilized (e.g., using ethylene oxide) and then tested for lubricity and durability (L&D). To test for L&D, a mandrel is inserted into the catheter shaft and the catheter shaft and mandrel secured within a clamping mechanism. A clamp was then closed about the catheter shaft at a normal force of about 800 grams (g). The force required to pull the catheter from the clamp was measured over a number of cycles. FIG. 4 illustrates a graph of the force required (in grams) to pull the catheter shaft from the clamp for each of the coating compositions. Each composition was tested without sterilization and with sterilization. As can be seen in FIG. 4, the sample having 0.05% of total solids of acrylic acid-acrylamide copolymer was the least lubricious and the least durable of the three samples. The sample having 1% of total solids of acrylic acid-acrylamide copolymer was the most lubricious and the most durable of the three samples. As described herein the lower MW PVP, higher MW PVP, and the acrylic acid-acrylamide copolymer are believed to be situated within the interstitial spaces of the cross-linked NPGDA. Acrylic acid-acrylamide copolymer absorbs more water than PVP. As such, increasing the percent of total solids of acrylic acid-acrylamide copolymer would be expected to cause the coating to become more slippery and more durable (e.g., due to less material sloughing off due to friction). However, unexpectedly, as the concentration of acrylic acid-acrylamide copolymer was increase to 5% of the total solids, the lubricity and durability of the coating was less than the coating having 1% of the total solids acrylic acid-acrylamide copolymer. Without wishing to be bound by theory, it believed that increasing concentrations of acrylic acid-acrylamide copolymer results in more water being absorbed and the acrylic acid-acrylamide copolymer “swelling out” of the interstitial spaces resulting in a rougher (e.g., less smooth) coating.

Although various embodiments are specifically illustrated and described herein, it will be appreciated that modifications and variations of the present disclosure are covered by the above teachings and are within the purview of the appended claims without departing from the spirit and intended scope of the invention.

What is claimed is:

1. A lubricous coating comprising (a) a higher molecular weight polyvinylpyrrolidone (PVP), (b) a lower molecular weight polyvinylpyrrolidone (PVP), (c) a copolymer of sodium acrylate and acrylamide or a copolymer of acrylic acid and acrylamide, and (d) a cross-linked polymer forming a semi-interpenetrating network.

2. The lubricous coating of claim 1, wherein the higher molecular weight PVP, the lower molecular weight PVP, and the copolymer of sodium acrylate and acrylamide or the copolymer of acrylic acid and acrylamide are positioned within interstitial spaces of the semi-interpenetrating network of the cross-linked polymer.

3. The lubricous coating of claim 1, wherein the higher molecular weight PVP has a K-value ranging from about 85 to about 95 and wherein the lower molecular weight PVP has a K-value ranging from about 15 to about 32.

4. The lubricous coating of claim 1, wherein the weight ratio of higher molecular weight polyvinylpyrrolidone to lower molecular weight polyvinylpyrrolidone ranges from about 70:30 to about 90:10.

5. The lubricous coating of claim 1, wherein the crosslinked polymer is formed from a polyfunctional ethylenically unsaturated monomer selected from a polyfunctional acrylate compound, a polyfunctional methacrylate compound and a polyfunctional vinyl compound.

6. The lubricous coating of claim 1, wherein the crosslinked polymer is formed from neopentyl glycol diacrylate.

7. The lubricous coating of claim 6, wherein the weight ratio of total polyvinylpyrrolidone to neopentyl glycol diacrylate ranges from about 0.5:1 to about 4:1.

8. A medical article comprising a medical article substrate that is at least partially covered by a layer of the lubricous coating in accordance with claim 1.

9. The medical article of claim 8, wherein the layer ranges from about 0.1 to about 20 micrometers in thickness.

10. The medical article of claim 8, wherein the medical article is an implantable or insertable medical device.

11. A lubricous coating composition comprising (a) a higher molecular weight polyvinylpyrrolidone (PVP), (b) a lower molecular weight polyvinylpyrrolidone (PVP), (c) a copolymer of sodium acrylate and acrylamide or a copolymer of acrylic acid and acrylamide, (d) a cross-linking agent, and (e) a solvent.

12. The lubricous coating composition of claim 11, wherein the solvent comprises isopropanol and water.

13. The lubricous coating composition of claim 12, wherein the weight ratio of water to isopropanol ranges from about 50:50 to about 10:90.

14. The lubricous coating composition of claim 11, wherein the total solids are adjustable to control a thickness of the coating and particulate shedding

15. The lubricous coating composition of claim 11, wherein the copolymer of sodium acrylate and acrylamide or the copolymer of acrylic acid and acrylamide comprises about 0.05 to about 10% of the total solids.

16. The lubricous coating composition of claim 11, further comprising a therapeutic agent.

17. A method comprising applying the lubricous coating composition of claim 11 to a substrate in the form of a layer, removing at least a portion of the solvent, and crosslinking the composition by applying of UV light.

18. The method of claim 17, further comprising sterilizing the substrate.

19. An article made by the method of claim 17, wherein the article is a medical article comprising a lubricious coating.

20. The article of claim 19, wherein the medical article is an implantable or insertable medical device.
